(19) United States
(12) Patent Application Publication
(10) Pub. No.: US 2026/0263284 A1
(43) Pub. Date: Sep. 10, 2026

(54) WOUND DRESSINGS WITH ADHESIVE BORDERS

(71) Applicant: PLASTOD S.P.A., Lippo di Calderara di Reno (BO) (IT)

(72) Inventor: Federico MELANDRI, Lippo di Calderara di Reno (BO) (IT)

(21) Appl. No.: 19/165,958

(22) PCT Filed: Mar. 13, 2024
(86) PCT No.: PCT/IB2024/052415

§ 371 Date: Sep. 16, 2025

(30) Foreign Application Priority Data
Mar. 17, 2023 (IT) 102023000005124

(51) Int. Cl. A61F 13/0246; A61F 13/00; A61L 15/18; A61L 15/24; A61L 15/26; A61L 15/28; A61L 15/58; A61L 15/60

(52) U.S. Cl. CPC A61F 13/0253; A61F 13/00987; A61L 15/18; A61L 15/24; A61L 15/26; A61L 15/28; A61L 15/58; A61L 15/60; A61L 2300/404

(57) ABSTRACT

Wound dressing (11) with adhesive borders (13) comprises a backing layer (15) located on the non-skin-facing surface of the dressing (11), a first adhesive layer (17) located on the skin-facing surface of the backing layer (15), a wound pad (19) attached to the backing layer (15), an inner layer (21) attached to the backing layer (15) and surrounding at least a portion of the wound pad (19), and a second adhesive layer (23) located on the skin-facing surface of the inner layer (21). The first adhesive layer (17) and the second adhesive layer (23) comprise different adhesive materials capable of exerting different adhesion strength to the skin of a human being.

### TECHNICAL FIELD

This invention relates to wound dressings with adhesive borders, The purpose of wound dressings with adhesive borders is to fix the wound pad to the wound via adhesion to the skin surrounding the wound.

Wound dressings with adhesive borders range from well-known patches for small and acute wounds up to those specially made for chronic wounds.

The main requirements for adhesive borders are:
they should allow the fixation of the wound dressing to the patient's skin to resist over a defined time, i.e., the wound dressing must remain in place without detaching (even partially) for a defined period, typically at least two to three days in case of small wounds and multiple days in case of chronic wounds;
they should allow breathability and vapor transmission, i.e., the material must be able to allow moisture vapor to be transmitted therethrough; they should be impermeable to bacteria and other microorganisms, i.e., the adhesive borders must not allow bacteria and other microorganisms to pass therethrough;
they should be waterproof, i.e., the material must be resistant to water;
they must allow atraumatic (painless) removal of the wound dressing.

Currently, fixation of the wound dressing is realized by coating the border with an adhesive. The most common adhesives are based on acrylate or silicone.

Regardless of the specific coating weights of the adhesive on the border, adhesive borders with acrylate adhesive have stronger adhesion to the skin than those with silicone adhesive, which naturally leads to longer fixation duration, less (punctual) de-tachment, as well as good protection against entering of water or bacteria beneath the border.

On the other hand, strong adhesion can be disadvantageous, as the change of the wound dressing can be painful (traumatic) and can lead to damage of the skin surrounding the wound.

On the contrary, silicone-coated adhesive borders are generally weaker in adhesion, which leads to subjectively higher wear comfort of the wound dressing. The removal of the wound dressing is less painful (traumatic) than with acrylate adhesives, and the risk of skin damaging is significantly lower.

Furthermore, silicone adhesives are known to be skin-friendly with less allergic in-tolerances than acrylate adhesives,

### BACKGROUND ART

EP 2 073 771 discloses a wound dressing comprising: a backing layer on the non-skin-facing surface of the dressing, an adhesive laminate on the skin-facing surface of the dressing, wherein said laminate comprises a first discontinuous acrylate-based adhesive layer distal to the skin, a support layer, a second continuous silicone-based adhesive layer, proximal to the skin-an absorbent foam layer, said foam layer being located between the backing layer and the adhesive laminate, and the foam layer being attached to the backing layer. Wound dressings constructed according to the teaching of this document comprise a silicone adhesive provided for fixation to the wound or the surrounding skin, and acrylate adhesive provided for construction of the wound dressing, i.e., for fixation of the individual layers and the wound pad.

With lowered adhesion strength exerted by silicone adhesives, the risk of potential detachments is generally higher, which could lead to leakages of water and bacteria underneath the border to the wound.

WO 2013 090810 describes a releasable medical drape comprising a perforated silicone layer overlying the wound, through the openings of which an acrylate layer disposed thereabove can penetrate to the wound.

The use of a perforated silicone layer does not avoid the risk of potential detachments and can also lead to the passage of water or bacteria under the medical drape. These foreign elements, passing through the areas where there is no contact between the skin and the acrylate layer, could come into contact with the wound.

The mentioned requirements are particularly relevant and essential in case of chronic wounds.

Chronic wounds include, but are not limited to, venous leg ulcers, diabetic ulcers, surgical wounds and pressure ulcers. Chronic leg and foot ulcers occur in many adults with vascular disease or diabetes mellitus and are becoming more common. Diabetic foot wounds are predominantly a result of diabetic neuropathy (nerve damage or de-generation) or peripheral vascular disease (poor blood supply because of disease of the large or medium sized blood vessels in the legs) or due to a combination of both disorders. Improving the clinical and economic benefits of non-healing complex wounds and preventing recurrence of healed ulcers is a key target for health services.

Chronic wounds are treated in a period of several weeks or months, until the wound is closed. Typically, the wound dressing applied on the wound is staying for multiple days until the dressing is changed. The wound needs to be protected from influences from outside (no entering of bacteria or other microorganisms, no entering of water, harmful particles etc.), must be breathable and sufficiently adherent to the surrounding skin and the change of wound dressing must be atraumatic.

### SUMMARY OF INVENTION

One object of the present invention is to provide a wound dressing capable of overcoming the drawbacks of the prior art.

Another object of the invention is to provide a wound dressing that is capable of resisting as long as possible attached to the patient's skin in a proper condition without significative reduction of performances, particularly in terms of impermeability to bacteria and other microorganisms, and that at the same time allows atraumatic (painless) removal of the wound dressing,

According to the invention, there is provided a wound dressing, which comprises adhesive borders and which can be advantageously used to treat both small wounds and acute and chronic wounds.

Advantageously, according to the invention, the adhesive borders combine the advantages of different adhesive materials by utilizing the border with both adhesives, preferably silicone and acrylate.

According to the invention, the surface of a first portion of the adhesive border is coated with silicone adhesive and the surface of a second portion of the adhesive border is coated with acrylate adhesive,

According to a preferred embodiment of the invention, the area of the first portion of the adhesive border is greater, more preferably largely greater than the area of the second portion.

According to a preferred embodiment of the invention, the second portion of the adhesive border coated with acrylate adhesive comprises the outer frame of the adhesive border around the whole or part of the wound pad.

The adhesive border can be continuous, i.e., it can surround the whole wound pad, or it can be interrupted, e.g., one or more sides of the wound pad can be free from adhesive border.

The outer frame of the adhesive border can be continuous, i.e., it can surround the whole adhesive border, or it can be interrupted and leave free the outer side of one or more portions of the adhesive border.

Preferably, the surface of the adhesive border is mostly coated with silicone adhesive, but the outer frame of the border around the wound pad is coated with acrylate adhesive. More particularly, in a preferred embodiment of the invention the ratio between the area of the second portion of the adhesive border and the area of the first portion of the adhesive border is set from 5:95 to 50:50.

The border can be based on thin, flexible and conformable polyurethane films.

### BRIEF DESCRIPTION OF DRAWINGS

The invention is disclosed more in detail with reference to the drawings, in which:

FIG. 1A shows a bottom plan view of a dressing of the invention in a first variant;

FIG. 1B shows a bottom plan view of a dressing of the invention in a second variant;

FIG. 2 shows an exploded view of the wound dressing according to FIG. 1;

FIG. 3 shows a cross-section of the wound dressing according to FIG. 1;

FIG. 4A shows a respective alternative shape of the wound dressing according to the invention;

FIG. 4B shows a respective alternative shape of the wound dressing according to the invention;

FIG. 4C shows a respective alternative shape of the wound dressing according to the invention.

### DESCRIPTION OF EMBODIMENTS

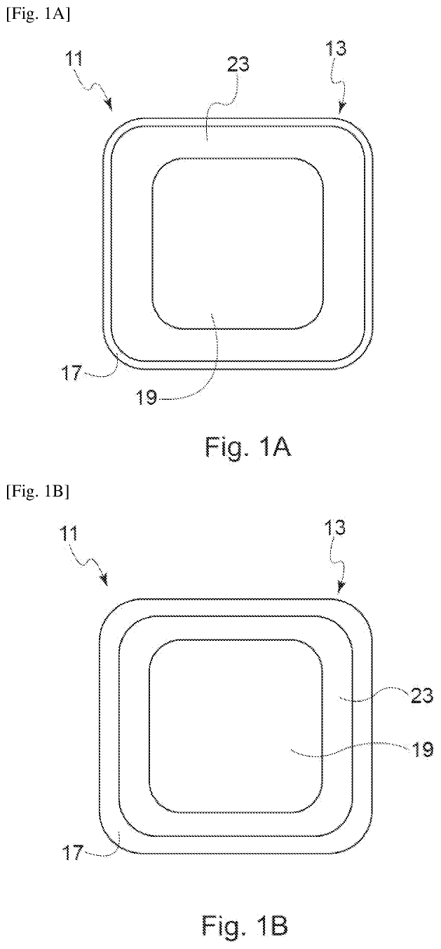

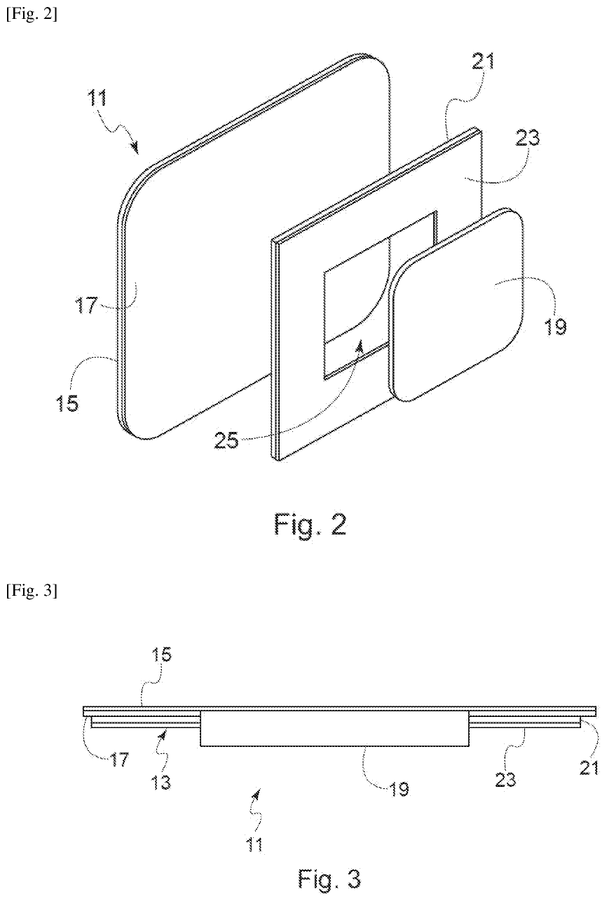

FIGS. 1A and 1B illustrate the wound dressing according to the invention as viewed at the wound contact side. Further description of the construction of the wound dressing according to the invention including the view at the cross section of the wound dressing is illustrated in FIGS. 2 and 3.

Referring in particular to FIGS. 1A and 1B, the invention relates to a wound dressing 11 with adhesive borders 13, said dressing comprising:
a backing layer 15 located on the non-skin-facing surface of the dressing 11,
a first adhesive layer 17 located on the skin-facing surface of the backing layer 15,
a wound pad 19 attached to the backing layer 15,
an inner layer 21 attached to the backing layer 15 and surrounding at least a portion of the wound pad 19,
a second adhesive layer 23 located on the skin-facing surface of the inner layer 21.

According to the invention, the first adhesive layer 17 and the second adhesive layer 23 comprise different adhesive materials capable of exerting different adhesion strength to the skin of a human being.

More particularly, according to the invention the first adhesive layer 17 comprises an adhesive material which exerts adhesion strength to the skin of a human being higher with respect to the adhesive material of the second adhesive layer 23.

Still more particularly, according to the invention, the first adhesive layer 17 comprises an acrylate-based adhesive material and the second adhesive layer 23 comprises a silicone-based adhesive material.

In a preferred embodiment of the invention, the ratio between the area of the adhesive skin-facing surface of the inner layer and the area of the adhesive skin-facing surface of the backing layer is set from 5:95(FIG. 1A) to 50:50(FIG. 1B).

The backing layer 15 and the inner layer 21 can comprise a film of polyurethane material.

In the illustrated example, the wound dressing 11 contains a wound pad 19, (“absorbent pad”), to absorb and retain wound exudate and to maintain a moist wound environment. The wound dressing 11 is breathable. The adhesive borders 13 have the purpose to adhere to the skin surrounding the wound.

In a typical non-limiting example embodying the invention, the area of the wound pad 19 is comprised between a minimum of 10.0×10.0 mm up to a maximum of 200.0×200.0 mm.

The wound dressing 11 can be squared, rectangular, triangular, oval or a combination of different geometries.

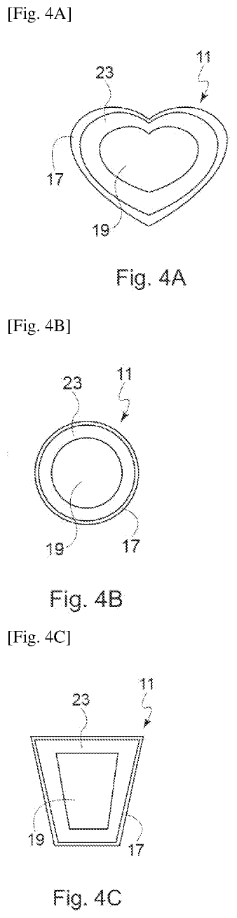

FIGS. 4A to 4C show respective alternative shapes of the wound dressing according to the invention; more particularly, FIG. 4A shows a fancy shape similar to a heart symbol, FIG. 4B shows a wound dressing having a circular shape and FIG. 4C shows a wound dressing having a trapezoidal shape.

In a typical non-limiting example, the width of the adhesive border is comprised between 30.0 mm and 280.0 mm.

The length of the adhesive border is 280.0 mm (minimum 30.0 mm). The adhesive border shall exceed at least 5.0 mm from the wound pad, but preferably shall not exceed 45.0 mm.

In a preferred embodiment, the wound pad 11 can be based on the following materials: polyurethane foam, non-woven (e.g., cellulose, viscose, PP, PE, etc.), superabsorbent pad/material. Still in a preferred embodiment, the wound pad has a thickness of ideally about 3.5 mm, but with a minimum of 1.0 mm and maximum of 7.0 mm.

Amongst other things, the performance of the wound pad can be measured by ab-sorption and retention values, and by their MVTR values, for example according to EN ISO 13726.

The wound pad can be based on one of the above materials or can be a combination of at least two of the above materials.

Example: superabsorbent pad/material in combination with a foam or superabsorbent pad/material in combination with a foam and a non-woven, whereby the wound contact site should be a non-woven or foam.

The materials of the wound pad can contain (activated) charcoal.

The wound pad can contain antimicrobial substances such as metals (copper, silver), organic compounds with biguanide functional groups such as PHMB or Chlorohexidine, (organo-)metal compounds or surfactants or, in combination, silicone dioxide ceramics.

The wound pad can contain gelling agents such as alginates.

The materials can have a thin layer at the wound contact site. The thin layer can be hydrophobic. The thin layer can be a perforated synthetic film such as polyurethane, polypropylene, polyester, PTFE and polyethylene.

Furthermore, the thin layer can be a mesh made of synthetic fabrics or cotton or a combination thereof.

The thin layer can be coated with silicone gel, or medical gel which contains Vaseline.

The thin layer can have antimicrobial or bacteriostatic properties. The thin layer can contain antimicrobial substances such as metals (copper, silver), organic compounds with biguanide functional groups such as PHMB or Chlorohexidine, (organo-)metal compounds or surfactants or, in combination, silicone dioxide ceramics, The adhesive borders 13 must be constructed with at least two films, a backing or outer film and an inner or second film attached to the outer film. The thickness of the films ranges from 15.0 to 100.0 μm. The film can be made of polyurethane materials, The inner film is coated with silicone adhesive at the wound contact site. The outer film at the wound contact site is coated with acrylate adhesive.

The inner film has a cut-out window 25. The wound pad 11 is attached to the outer and inner film, covering the cut-out window 25.

The inner film can be perforated.

The adhesive border is breathable, waterproof and impermeable to microbials.

The surface of the adhesive border of the wound dressing contains at least two different adhesive areas, based on acrylate and silicone. The area of the skin contact site must cover preferably a minimum of 5% with acrylate adhesive and a maximum of 95% with silicone adhesive. Preferably, the maximum coverage of the skin contact surface of the acrylate adhesive is 50% and the minimum coverage of the skin contact surface with silicone adhesive is 50%.

In a preferred embodiment of the invention, the skin contact surface area towards the margin of the adhesive borders is always acrylate adhesive.

The coating weight specification of the acrylate adhesive is 10-80 g/m2. The coating weight specification of the silicone adhesive is 40-180 g/m2.

According to another aspect of the present invention, a method of manufacturing a wound dressing 11 with adhesive borders is provided.

Said method includes the following steps:
providing the backing layer 15 intended for being located on the non-skin-facing surface of the dressing 11 when the wound dressing is in use,
providing the first adhesive layer 17 on a surface of the backing layer 15 intended to face the skin when the wound dressing 11 is in use,
providing the wound pad 19,
attaching said wound pad 19 to said first adhesive layer 17,
providing the inner layer 21,
attaching said inner layer to said first adhesive layer 17 so as to surround at least a portion of the wound pad 19,
providing the second adhesive layer 23 on the skin-facing surface of the inner layer 21 when the wound dressing is in use,
providing said first adhesive layer 17 and said second adhesive layer 23 by means of different adhesive materials capable of exerting different adhesion strength to the skin of a human being.

The above description of a preferred embodiment of the invention has been given merely by way of non-limiting example, and several modifications and variations within the reach of the person skilled in the art are possible without departing from the scope of the invention as defined by the appended claims.

1. A wound dressing (11) with adhesive borders (13), said dressing comprising:
a backing layer (15) located on the non-skin-facing surface of the dressing (11),
a first adhesive layer (17) located on the skin-facing surface of the backing layer (15),
a wound pad (19) attached to the backing layer (15),
an inner layer (21) attached to the backing layer (15) and surrounding at least a portion of the wound pad (19), and
a second adhesive layer (23) located on the skin-facing surface of the inner layer (21), characterized in that the first adhesive layer (17) and the second adhesive layer (23) comprise different adhesive materials capable of exerting different adhesion strength to the skin of a human being.

2. The wound dressing (11) according to claim 1, wherein the first adhesive layer (17) comprises an adhesive material which exerts adhesion strength to the skin of a human being higher with respect to the adhesive material of the second adhesive layer (23).

3. The wound dressing (11) according to claim 2, wherein the first adhesive layer (17) comprises an acrylate-based adhesive material and the second adhesive layer (23) comprises a silicone-based adhesive material.

4. The wound dressing (11) according claim 1, wherein the surface of the adhesive borders (13) of the wound dressing contains at least two different adhesive areas, based respectively on acrylate and silicone adhesive materials.

5. The wound dressing (11) according to claim 4, wherein the area of the skin contact side of the adhesive borders (13) is covered by a minimum of 5% with acrylate adhesive and a maximum of 95% with silicone adhesive.

6. The wound dressing (11) according to claim 5, wherein the maximum coverage of the skin contact side of the acrylate adhesive is 50% and wherein the minimum coverage of the skin contact side of the silicone adhesive is 50%.

7. The wound dressing (11) according to claim 6, wherein the perimetral area of the skin contact side of the adhesive borders comprises acrylate adhesive.

8. The wound dressing (11) according to claim 7, wherein said wound pad (19) is based on polyurethane foam, non-woven cellulose, viscose, PP, PE, superabsorbent pad/material, or a combination of at least two of said materials.

9. The wound dressing (11) according to claim 8, wherein said materials that compose said wound pad (19) have a layer at the wound contact site, wherein said thin layer may be selected from the group consisting of: a perforated synthetic film of polyurethane, polypropylene, polyester, PTFE or polyethylene; a mesh made of synthetic fabrics or cotton or a combination thereof;
wherein said thin layer may also comprise:
a coating of silicone gel or medical gel;
antimicrobial substances consisting of the group selected from copper, silver, organic compounds with biguanide functional groups (organo-)metal compounds and surfactants or, in combination, silicone dioxide ceramics.

10. A method of manufacturing a wound dressing (11) with adhesive borders according to claim 7, wherein the method comprises the steps of:
providing a backing layer (15) intended for being located on the non-skin-facing surface of the dressing (11) when the wound dressing is in use,
providing a first adhesive layer (17) on a surface of the backing layer (15) intended to face the skin when the wound dressing (11) is in use,
providing a wound pad (19),
attaching said wound pad (19) to said first adhesive layer (17),
providing an inner layer (21),
attaching said inner layer to said first adhesive layer (17) so as to surround at least a portion of the wound pad (19),
providing a second adhesive layer (23) on the skin-facing surface of the inner layer (21) when the wound dressing is in use, characterized in that it further comprises the steps of:
providing said first adhesive layer (17) and said second adhesive layer

(23) by means of different adhesive materials capable of exerting different adhesion strength to the skin of a human being.

11. The wound dressing (11) according to claim 4, wherein the maximum coverage of the skin contact side of the acrylate adhesive is 50% and wherein the minimum coverage of the skin contact side of the silicone adhesive is 50%.

12. The wound dressing (11) according to claim 1, wherein the perimetral area of the skin contact side of the adhesive borders comprises acrylate adhesive.

13. The wound dressing (11) according to claim 1, wherein said wound pad (19) is based on polyurethane foam, non-woven cellulose, viscose, PP, PE, superabsorbent pad/material, or a combination of at least two of said materials.

14. The wound dressing (11) according to claim 13, wherein said materials that compose said wound pad (19) have a layer at the wound contact site, wherein said thin layer may be selected from the group consisting of: a perforated synthetic film; a mesh made of synthetic fabrics or cotton or a combination thereof;
wherein said thin layer may also comprise:
a coating of silicone gel or medical gel;
antimicrobial substances of metals, organic compounds with biguanide functional groups, (organo-)metal compounds or surfactants or, in combination, silicone dioxide ceramics.

15. A method of manufacturing a wound dressing (11) with adhesive borders according to claim 1, wherein the method comprises the steps of:
providing a backing layer (15) intended for being located on the non-skin-facing surface of the dressing (11) when the wound dressing is in use,
providing a first adhesive layer (17) on a surface of the backing layer (15) intended to face the skin when the wound dressing (11) is in use,
providing a wound pad (19),
attaching said wound pad (19) to said first adhesive layer (17),
providing an inner layer (21),
attaching said inner layer to said first adhesive layer (17) so as to surround at least a portion of the wound pad (19),
providing a second adhesive layer (23) on the skin-facing surface of the inner layer (21) when the wound dressing is in use, characterized in that it further comprises the steps of:
providing said first adhesive layer (17) and said second adhesive layer (23) by means of different adhesive materials capable of exerting different adhesion strength to the skin of a human being.
